(19) United States
(12) Patent Application Publication
(10) Pub. No.: US 2012/0275772 A1
(43) Pub. Date: Nov. 1, 2012

(54) Rotating Electrical Plug Assembly for Volatile Material Dispenser

(76) Inventor: David C. Belongia, Burlington, WI (US)

(21) Appl. No.: 13/096,767

(22) Filed: Apr. 28, 2011

(51) Int. Cl. A61M 16/00

(52) U.S. Cl. 392/390

(57) ABSTRACT

A support block for a rotatable plug assembly includes a base and electrical plug pins extending outwardly therefrom. A medial wall extends from the base. A cap is hingedly attached to the medial wall and is moveable between open and closed positions. A sidewall extends from the medial wall and has at least one notch disposed therein. At least one electrical wire is connected to the support block and extends through the at least one notch. The cap fixedly retains the at least one electrical wire within the at least one notch when in the closed position.

### CROSS REFERENCE TO RELATED APPLICATIONS

Not applicable.

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Invention

The present invention relates generally to a rotating electrical plug assembly and, more particularly, to a rotating electrical plug assembly for use in a volatile material dispenser.

2. Description of the Background of the Invention

Various volatile material dispensers are known in the prior art and generally include a housing with a refill inserted therein. The refill generally includes a container for holding a volatile material therein. In some dispensers, the volatile material is passively emitted therefrom. In other dispensers, a diffusion element is utilized to facilitate the dispensing of the volatile material. Examples of diffusion elements include heaters, piezoelectric elements, fans, aerosol actuators, and the like. Regardless of the manner in which the volatile material is emitted, once the volatile material has been expended from the refill, the refill is removed by a user and replaced with a new refill.

One type of volatile material dispenser, referred to herein as a plug-in scented oil dispenser, includes a housing and a heater disposed within the housing. A refill for use with a plug-in scented oil dispenser generally includes a container having a wick and a volatile material disposed within the container. Upon insertion of the refill into the dispenser, at least a portion of the wick is disposed adjacent the heater such that volatile material that moves through the wick is volatilized by the heater. The volatile material dispenser typically includes a plug assembly having electrical prongs extending outwardly from the housing. The electrical prongs are inserted into a standard electrical outlet and thereafter supply electrical energy to the volatile material dispenser.

One of the disadvantages of many of the volatile material dispensers and refills therefore, such as the plug-in scented oil dispenser discussed above, is that the plug assembly and electrical prongs are oriented in such a way so as to be compatible with only a single orientation of the volatile material dispenser with respect to the electrical outlet. For example, the vertical orientation of the electrical prongs with respect to the volatile material dispenser only allows a user of the volatile material dispenser to insert the dispenser in an upright manner into a vertically oriented electrical outlet. The user must rotate the volatile material dispenser in a horizontal manner to insert the volatile material dispenser into a horizontally oriented electrical outlet. Rotation of the dispenser in this manner is undesirable for numerous reasons including, at least, that the volatile material may leak from the dispenser and/or be removed from contact with the wick if the dispenser is disposed in a horizontal position.

A solution has been attempted to try to remedy the aforementioned problem in the form of a rotating plug assembly, which allows the rotation of the electrical prongs on the plug assembly from a vertically oriented position to a horizontally oriented position. The user may then adjust the plug assembly based on the desired orientation, which allows the volatile material dispenser to remain upright, while still receiving electrical energy. However, a further problem is created by the continued rotation of the plug assembly of the present devices utilizing such an assembly. Specifically, strain is created on various portions of the plug assembly during rotation, which causes the plug assembly parts to fatigue over time. The fatigue may lead to loose wires in the plug assembly that could create unstable connection points between the plug assembly and the wires and render the volatile material dispenser inoperable.

### SUMMARY

According to one aspect of the present invention, a support block for a rotatable plug assembly includes a base and electrical plug pins extending outwardly therefrom. A medial wall extends from the base. A cap is hingedly attached to the medial wall and is moveable between open and closed positions. A sidewall extends from the medial wall and has at least one notch disposed therein. At least one electrical wire is connected to the support block and extends through the at least one notch. The cap fixedly retains the at least one electrical wire within the at least one notch when in the closed position.

In a different aspect of the present invention, a housing for a volatile material dispenser includes a front face and a rear face that form a chamber therebetween for receipt of a container. A support block is rotatably retained within an opening in the rear face. The support block includes an impingement member and a sidewall with at least one notch disposed therein. At least one electrical wire is connected to the support block and extends through the at least one notch. The impingement member fixedly retains the at least one electrical wire within the at least one notch.

In a further aspect of the present invention, a volatile material dispenser includes a container with a volatile material therein and a housing adapted to releasably retain the container. A support block and plug assembly are rotatably disposed within the housing. The support block includes a base and a cap attached thereto. The base includes a sidewall having at least one notch disposed therein and the cap includes an impingement member extending therefrom.

### BRIEF DESCRIPTION OF THE DRAWINGS

FIG. 1 is a front isometric view of a volatile material dispenser having a container disposed within a housing;

FIG. 2 is a front isometric view of the container of FIG. 1;

FIG. 3 is a side isometric view of the housing of FIG. 1;

FIG. 4 is a rear isometric exploded view of the housing of FIG. 1 with a support block removed therefrom;

FIG. 5 is a rear isometric view of the housing of FIG. 1 having an electrical plug assembly in a first position;

FIG. 6 is a rear isometric view of the housing of FIG. 1 having an electrical plug assembly in a second position;

FIG. 7 is a top isometric view of the electrical plug assembly of FIG. 5;

FIG. 8 is a side isometric view of the electrical plug assembly of FIG. 7;

FIG. 9 is a top isometric view of the electrical plug assembly of FIG. 5 further showing a cap in an open position and electrical wires extending therefrom and terminating at a heater;

FIG. 10 is a top plan view of the electrical plug assembly of FIG. 9 with the cap in a closed position; and

FIG. 11 is a cross-sectional view of the electrical plug assembly of FIG. 10 taken along the lines 11-11 in FIG. 10.

Other aspects and advantages of the present invention will become apparent upon consideration of the following detailed description, wherein similar structures have like or similar reference numerals.

### DETAILED DESCRIPTION

The present invention is directed to an electrical plug assembly for a volatile material dispenser. While the present invention may be embodied in many different forms, several specific embodiments are discussed herein with the understanding that the present invention is to be considered only as an exemplification of the principles of the invention, and it is not intended to limit the invention to the embodiments illustrated.

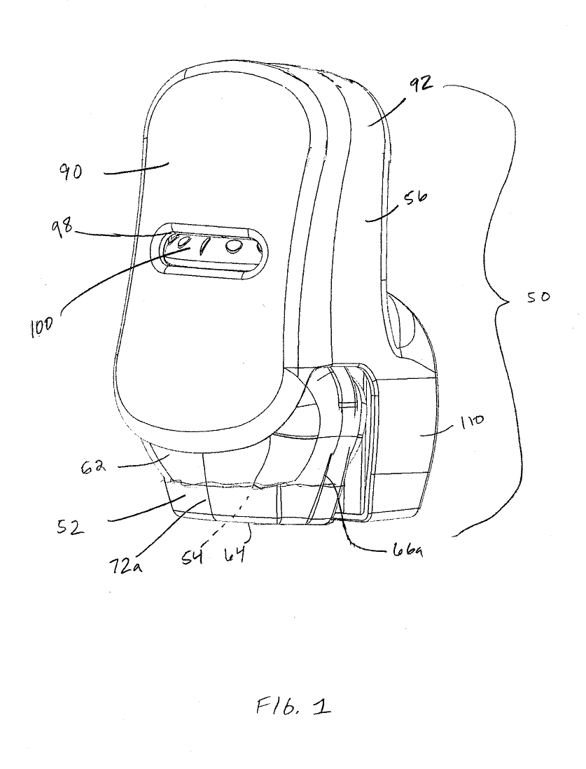

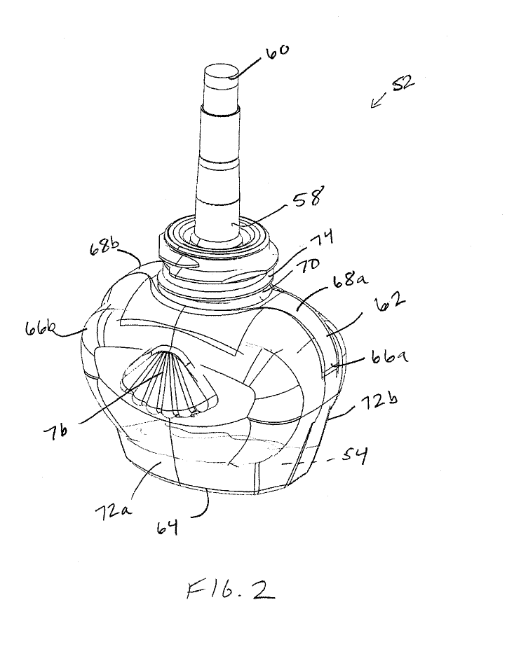

Referring to the drawings, FIG. 1 depicts a volatile material dispenser 50 having a container 52 with a volatile material 54 therein, which is adapted to be retained by a housing 56. As best seen in FIG. 2, the container 52 includes a retaining mechanism 58 to hold a wick 60 within the container 52. The container 52 includes a body 62 with the volatile material 54 disposed therein. The body 62 includes a base portion 64 and first and second opposing sidewalls 66a, 66b that extend upwardly and outwardly prior to curving inwardly toward first and second top walls 68a, 68b, respectively. The first and second top walls 68a, 68b are integral with a neck 70. Similarly, third and fourth opposing front and rear walls 72a, 72b, respectively, curve upwardly toward the neck 70.

The neck 70 includes a threaded portion 74 disposed on an outer surface thereof and an opening (not shown) disposed through a top portion thereof, wherein the opening allows access to the volatile material 54. The container 52 further optionally includes raised portions 76 extending outwardly from one or more of the third and fourth opposing front and rear walls 72a, 72b. In the present embodiment, the raised portions 76 are in the form of inverted shell-shaped members. Although a specific container 52 is described with particularity, it is contemplated that any type of container may be used with the electrical plug assembly described herein. For example, containers useful for the present invention include the containers described in U.S. Pat. No. 7,032,831 filed on Mar. 21, 2003, and the containers described in U.S. patent application Ser. No. 12/969,261 filed on Dec. 15, 2010, both of which are owned by the same assignee as the present invention.

The volatile material 54 disposed in the container 52 may be any type of volatile material adapted to be dispensed into an environment. For example, the container 52 may include a cleaner, an insecticide, an insect repellant, an insect attractant, a disinfectant, a mold or mildew inhibitor, a fragrance, a disinfectant, an air purifier, an aromatherapy scent, an antiseptic, an odor eliminator, a positive fragrancing volatile material, an air-freshener, a deodorizer, or the like, and combinations thereof. Additives may be included in the volatile material, such as, for example, fragrances, and/or preservatives.

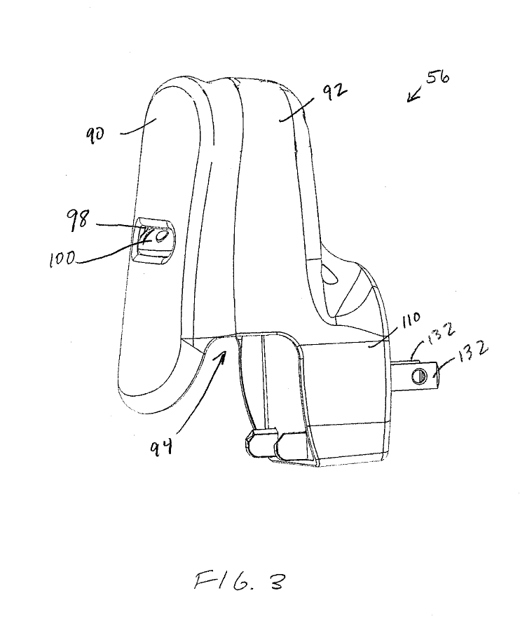

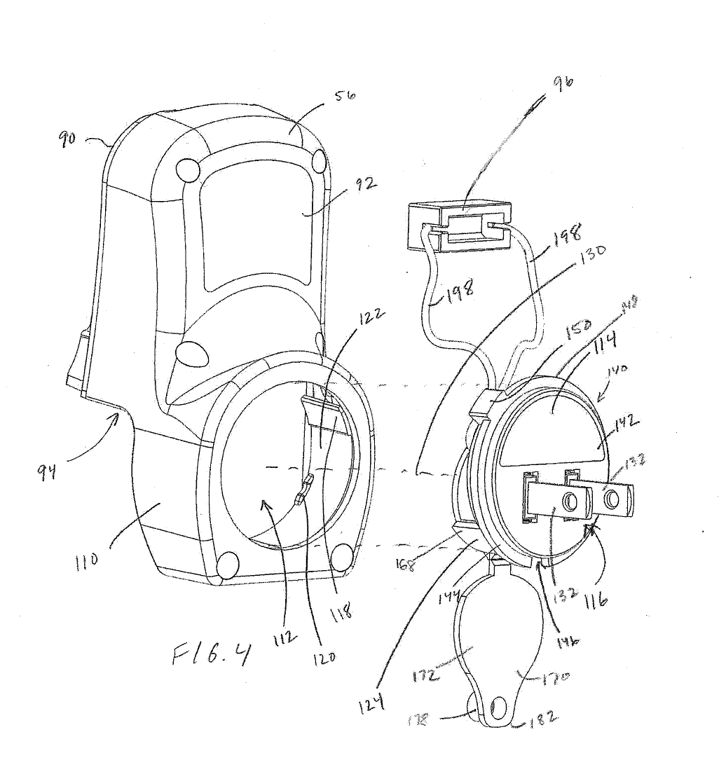

Now turning generally to FIGS. 1 and 3, the housing 56 of the volatile material dispenser 50 includes a slightly curved front face 90 attached to a curvilinear rear face 92 to form a chamber 94 (see FIG. 3) therebetween. The container 52 is inserted into the housing 56 by inserting the wick 60 thereof upwardly into the chamber 94. The container 52 is retained within the housing 56 by conventionally known means, including a snap-fit connection, a threaded interaction, and the like. Preferably, a portion of the wick 56 is disposed adjacent a heater 96 (see FIG. 4) that is disposed in the housing 56. The front face 90 of the housing 56 optionally includes an opening 98 with a control mechanism 100 extending partially therethough, which in the present embodiment comprises a rotatable dial adapted to control operational parameters of the volatile material dispenser 50.

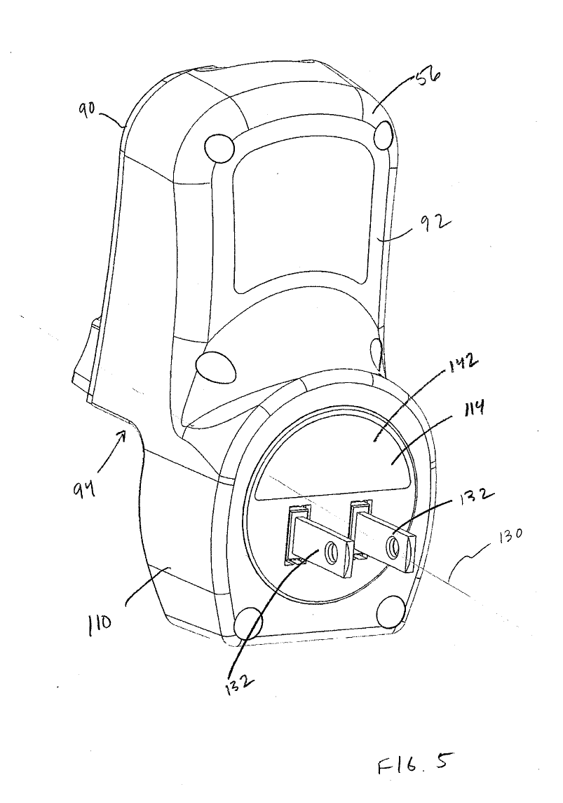

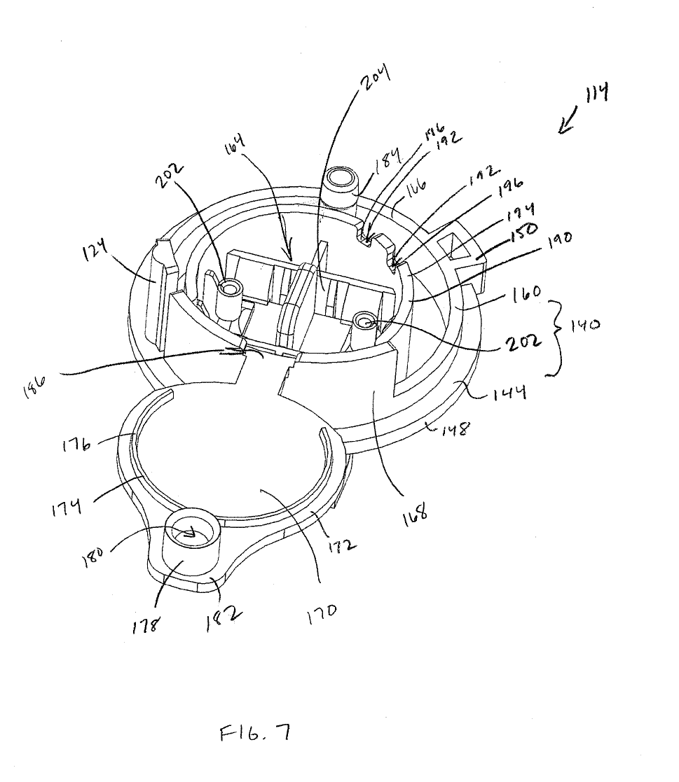

As best seen in FIGS. 3 and 4, the rear face 92 of the housing 56 includes a bulbous lower end 110 extending outwardly therefrom. The lower end 110 includes a circular opening 112 adapted to receive a support block 114 that provides a base for an electrical plug assembly 116. Two stop flanges 118 (only one shown) and two stop members 120 (only one shown) are provided on an internal sidewall 122, which defines a portion of the opening 112. The stop members 120 includes grooves that interact with a corresponding flange 124 (see FIGS. 7-10) located on the support block 114 to prevent inadvertent rotation of the electrical plug assembly 116 when provided in one of a first or second position (see FIGS. 5 & 6). The support block 114 may also be held in place by deformable tabs (not shown), or by any other conventional attaching means, which enables the support block 114 to be fixedly retained within the opening 112.

When the container 52 is inserted into the housing 56 (see FIG. 1), the top of the wick 60 extends upwardly through the chamber 94 such that a portion of the wick 60 is disposed adjacent the heater 96. The container 52 is releasably retained within the housing 56 by conventionally known manners. The positioning of the wick 60 with respect to the heater 96 enables the heat generated by the heater or heating element to assist in vaporizing the volatile material 54 drawn up through the wick 60 and to thereby release the volatile material into the surrounding atmosphere.

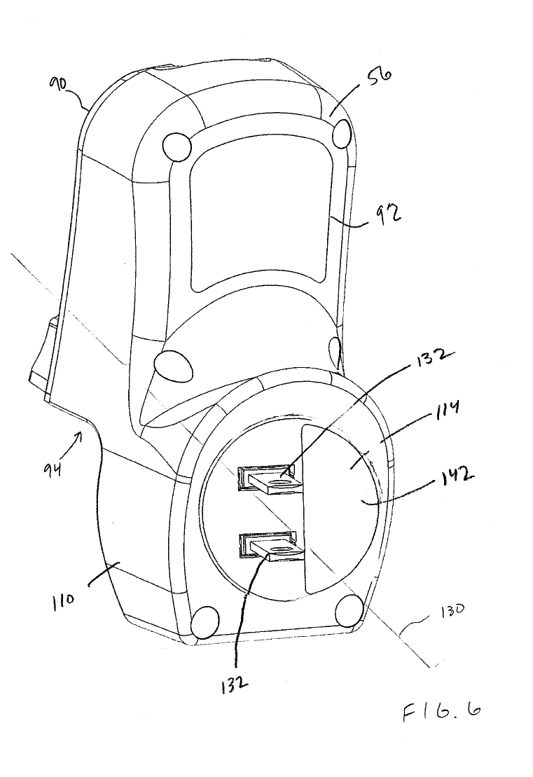

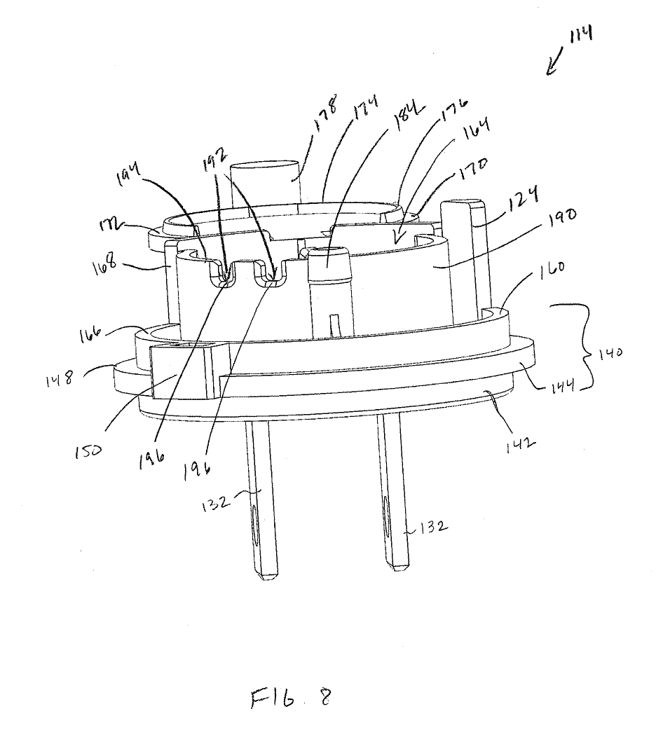

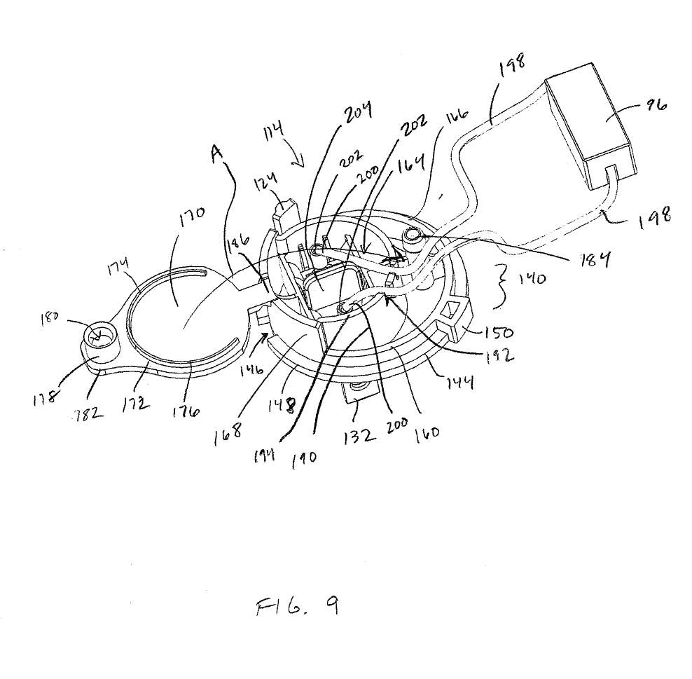

As shown in FIGS. 5 and 6, support block 114 can be rotated approximately 90 degrees around an axis 130 parallel to electrical plug pins 132 from a first position (see FIG. 5) to a second position (see FIG. 6). The rotational capabilities enable the volatile material dispenser 50 to be plugged into a conventional electric outlet so that the wick 60 is approximately perpendicular to the ground. As best seen in FIGS. 7 and 8, the electrical plug pins 132 extend outwardly from a stepped circular surface 140 of the support block 114. The stepped circular surface 140 includes a base 142, in which the pins 132 extend outwardly from, and a medial circular wall 144 having a slightly larger circumference than the base 142 integrally attached thereto. The medial wall 144 is truncated by a notch 146 (see FIG. 9) disposed in an edge 148 of the wall 144 and further includes a stop member 150 that is spaced away from the notch 146 and protrudes outwardly from the edge 148. The stop member 150 interacts with the top flanges 118 (see FIG. 4) within the housing 56 to prevent over-rotation of the support block 114 between the first and second positions.

As best seen in FIGS. 7 and 8, a generally circular sidewall 160 is spaced from the edge 148 of the medial wall 144 and extends upwardly therefrom. The sidewall 160 defines a compartment 164 adapted to contain various portions of the electrical plug assembly 116 as explained in more detail hereinbelow. The sidewall 160 includes a first segment 166 that extends circumferentially around a majority of the medial wall 160 and terminates at a support wall 168. The support wall 168 has a height component that is greater than the first segment 166 of the sidewall 160. The support wall 168 acts as an attachment point for an integral cap 170 adapted to enclose the compartment 164.

The cap 170 is hingedly attached to the support wall 168 and includes a base portion 172 having an impingement member extending therefrom. In one embodiment, the impingement member is provided in the form of a semi-circular rib 174. In a different embodiment, the impingement member may be any shape and/or size that extends from the cap 170 and/or other portion of the support block 114, so long as the impingement member provides a diffusing function of the rotational forces that act upon portions of the support block 114 as described in more detail hereinbelow. In the present embodiment, the rib 174 extends partially around the circumference of the base portion 172 and includes a flat upper surface 176 along the length thereof. The rib 174 is adapted to assist in securing portions of the electrical plug assembly 116 in a manner such that the rotational forces that act on the electrical plug assembly 116 are dispersed therethrough. The cap 170 further includes a cylindrical sidewall 178 having an orifice 180 therein and which is disposed at an end 182 thereof. The sidewall 178 is adapted to interact with a corresponding projection 184 disposed on and extending upwardly from the medial wall 144. The cap 170 is releasably secured to the medial wall 144 by rotating the cap 170 about a hinged attachment point 186 in a manner as shown by arrow A (see FIG. 9). After rotation, the projection 184 is inserted into the orifice 180 of the sidewall 178 to secure the cap 170 thereto.

Still referring to FIGS. 7-9, a semi-circular sidewall 190 extends outwardly from the support wall 168 and includes at least one notch 192 disposed in a top edge 194 thereof. Two notches 192 are shown in the present embodiment. However, it is contemplated that any number of notches may be included in the sidewall 190. The notches 192 each include a rounded edge 196 that acts as a support surface for at least one electrical wire 198. In the present embodiment, two electrical wires 198 are provided, which are connected at first ends 200 to crimp terminals 202 disposed within the chamber 164. The electrical wires 198 are disposed adjacent a pin bridge 204 that is disposed centrally within the chamber 164 and assists in controlling the electric power that is supplied to the volatile material dispenser 50. An electric circuit or other electronic components for use in the operation of the dispenser may also be included. The electrical wires 198 extend outwardly from the crimp terminals 202 and through the notches 192 before terminating at the heater 96. It should be apparent that any number of wires and notches may be provided as needed in the volatile material system, but it is preferable to include an equivalent number of notches and wires.

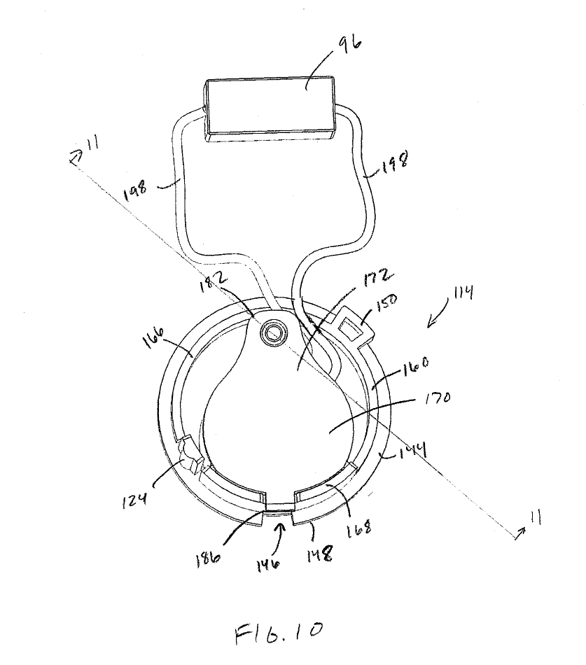

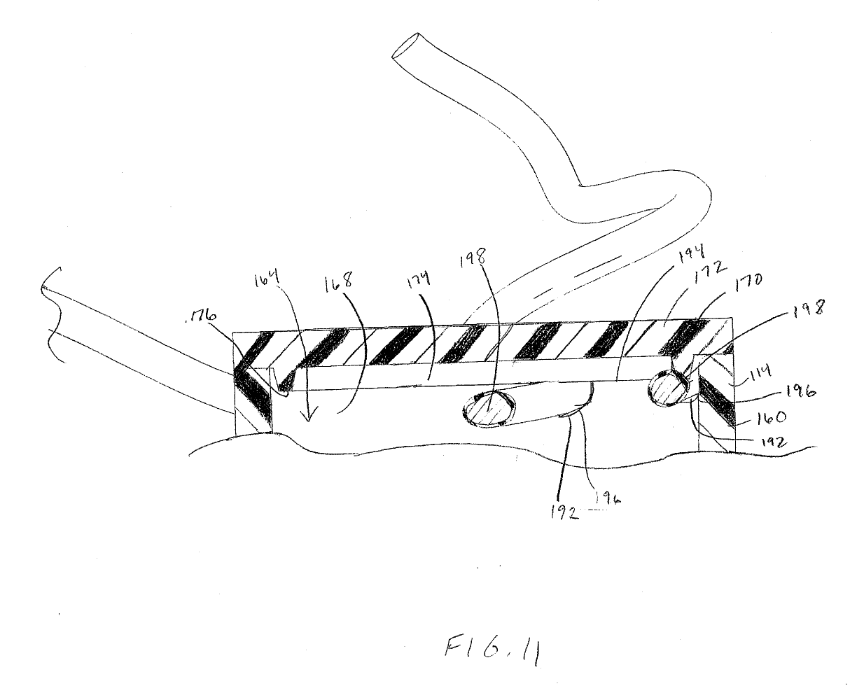

The support block 114 and the cap 170 are preferably manufactured as an integral piece and hingedly attached as described herein. As best seen in FIGS. 10 and 11, the cap 170 of the support block 114 is preferably closed prior to the support block 114 being inserted into the opening 112 of the housing 56. Closing the cap 170 causes the top surface 176 of the rib 174 on the base portion 172 of the cap 170 to press downwardly on the electrical wires 198 to fixedly secure the wires 198 in the notches 192 in the sidewall 190 (see FIG. 11). The contact point between the rib 174 and the wires 198 is important in realizing the advantages of the electrical plug assembly 116 described herein. Specifically, when the support block 114 is rotated between a first position (shown in FIG. 5) to a second position (shown in FIG. 6), the rotational forces that act on the wires 198 are diffused outwardly through the rib 174 on the cap 170 due to the substantially fixed nature of the wires 198 being pressed by portions of the cap 170 and portions of the sidewall 190 defining the notches 192. In conventional plug assemblies, the rotational forces act on the first ends 200 of the wires 198, which cause the wires 198 to become loose at the crimp terminals 202, thereby creating an unstable connection point for the wires. The rib 174/wire 198 interaction disclosed herein provides strain relief and substantially prevents the rotational forces from acting on the first ends 200 of the wires 198, which ultimately allows for increased rotational capabilities. The rotational forces that act upon the wires 198 are consolidated and diffused at the interaction point between the impingement member and wires 198, which minimizes the rotational forces felt on the first ends 200 of the wires 198 adjacent the crimp terminals 202. The integrally molded cap 170 also provides electrical insulation for the crimp terminals 202.

Although the dispensers for which the refills herein are described as being used with are plug-in scented oil dispensers, such refills may be utilized with any type of dispenser. In particular, the dispensers employing the refills described herein may comprise a variety of forms as known in the art. The dispensers also optionally may include one or more of a heater, a fan, and/or other components disposed in a housing that help facilitate the release of the volatile active. Any of the aforementioned components may be electrically connected to the support block in manners described herein.

The dispensers may further include one or more openings in the housing to allow for the volatile material to be dispensed from the housing to the surrounding environment. The housing may include a variety of internal implements to help secure the various refill bottles disclosed herein, such as, for example, snaps, ridges, undercuts, lips, notches, and/or other attachment methods. The dispenser may optionally include one or more refills and may operate using a variety of timing sequences as known in the art.

Any of the embodiments described herein may be modified to include any of the structures or methodologies disclosed in connection with other embodiments.

All documents cited in the Detailed Description of the Invention are, in relevant part, incorporated herein by reference; the citation of any document is not to be construed as an admission that it is prior art with respect to the present invention.

### INDUSTRIAL APPLICABILITY

The present invention provides a volatile material dispenser having a rotating electrical plug assembly. The plug assembly includes a cap integrally molded with a support block, which is adapted to support electrical wires. The wires are disposed within notches and held therein by a rib that extends outwardly from the cap. The connection point between the rib and the wires allows for strain to be dissipated as opposed to acting on ends of the wires.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

I claim:

1. A support block for a rotatable plug assembly, comprising:
a base and electrical plug pins extending outwardly therefrom;
a medial wall extending from the base and further including a cap hingedly attached thereto, which is moveable between open and closed positions;
a sidewall extending from the medial wall and having at least one notch disposed therein; and
at least one electrical wire connected to the support block and extending through the at least one notch, wherein the cap fixedly retains the at least one electrical wire within the at least one notch when in the closed position.

2. The rotatable plug assembly of claim 1, wherein the support block is rotatable from a first position in which the electrical plug pins are oriented in a vertical manner to a second position in which the electrical plug pins are oriented in a horizontal manner.

3. The rotatable plug assembly of claim 1, wherein the cap is integral with the medial wall.

4. The rotatable plug assembly of claim 1, wherein the cap includes a rib extending therefrom, wherein the rib is in physical communication with the at least one electrical wire when the cap is in a closed position to fixedly retain the at least one electrical wire within the at least one notch.

5. The rotatable plug assembly of claim 1, wherein the at least one notch is disposed in an upper edge of the sidewall.

6. The rotatable plug assembly of claim 1, wherein crimp terminals are provided on the base and are adapted to support first ends of the electrical wires attached thereto.

7. The rotatable plug assembly of claim 1, wherein first and second notches are provided within the sidewall for receipt of first and second electrical wires, respectively.

8. A housing for a volatile material dispenser, comprising:
a front face and a rear face that form a chamber therebetween for receipt of a container;
a support block rotatably retained within an opening in the rear face, wherein the support block includes an impingement member and a sidewall with at least one notch disposed therein; and
at least one electrical wire connected to the support block and extending through the at least one notch, wherein the impingement member fixedly retains the at least one electrical wire within the at least one notch.

9. The housing of claim 8, wherein the impingement member is disposed on a cap attached to the support block.

10. The housing of claim 9, wherein the cap is attached to the support block by a hinge.

11. The housing of claim 8, wherein the impingement member is provided in the form of a rib.

12. The housing of claim 11, wherein rotational strain is dissipated transversely through the rib when the support block is rotated from a first position to a second position.

13. The housing of claim 12, wherein the rotational strain on the at least one electrical wire is greater at a point adjacent where the rib contacts the at least one electrical wire in comparison to the rotational strain on the at least one electrical wire adjacent a crimp terminal of the at least one electrical wire.

14. The housing of claim 8 further including a heating element in the form of a heater electrically connected to the support block.

15. A volatile material dispenser, comprising:
a container having a volatile material therein;
a housing adapted to releasably retain the container; and
a support block and plug assembly rotatably disposed within the housing, wherein the support block includes a base and a cap attached thereto, and wherein the base includes a sidewall having at least one notch disposed therein and the cap includes an impingement member extending therefrom.

16. The volatile material dispenser of claim 15 further including a wick disposed at least partially within the container and contacting the volatile material.

17. The volatile material dispenser of claim 16 further including a heating element in the form of a heater disposed within the housing and electrically connected to the rotatable plug assembly with at least one electrical wire.

18. The volatile material dispenser of claim 17, wherein the heater is disposed adjacent a portion of the wick when the container is disposed within the housing.

19. The volatile material dispenser of claim 17, wherein the impingement member of the cap contacts the at least one electrical wire to retain same within the at least one notch.

20. The volatile material dispenser of claim 19, wherein rotational strain is dissipated transversely through the impingement member when the rotatable plug assembly is rotated from a first position to a second position.
